(19) United States
(12) Patent Application Publication
(10) Pub. No.: US 2004/0198653 A1
(43) Pub. Date: Oct. 7, 2004

(54) Pharmaceutical compositions which inhibit proliferation of pituitary adenomas and method of use thereof

(76) Inventors: Michael De Witt Culler, Hopkinton, MA (US); Ettore C Degli Uberti, Ferrara (IT); Maria C Zatelli, Ferrara (IT)

Correspondence Address: Brian Morill, Biomeasure, 27 Maple Street, Milford, MA, 01757, US

(21) Appl. No.: 10/481,066

(22) PCT Filed: Jun. 25, 2002
(86) PCT No.: PCT/US02/19998

Related U.S. Application Data
(60) Provisional application No. 60/300,909, filed on Jun. 25, 2001.

(51) Int. Cl. A61K 38/17

(52) U.S. Cl. 514/12

(57) ABSTRACT

The present invention is directed to a method of reducing the rate of proliferation of adenoma cells which method comprises contacting said pituitary adenoma cells with one or more of an SSTR1 agonist, and/or one or more of an SSTR2 agonist, and/or one or more of SSTR5 agonist, or one or more pharmaceutically acceptable salts thereof, either alone or in combination.

### BACKGROUND OF THE INVENTION

Somatostatin (SRIF), a tetradecapeptide discovered by Brazeau et al., has been shown to have potent inhibitory effects on various secretory processes in tissues such as pituitary, pancreas and gastrointestinal tract. SRIF also acts as a neuromodulator in the central nervous system. These biological effects of SRIF, all inhibitory in nature, are elicited through a series of G protein coupled receptors, of which five different subtypes have been characterized (SSTR1-SSTR5) (Reubi J C, et al., Cancer Res 47: 551-558, Reisine T, et al., Endocrine Review 16: 427-442, Lamberts S W, et al., Endocr Rev 12: 450-482, 4 Patel Y C, 1999 Front Neuroendocrinology 20: 157-198). These five subtypes have similar affinities for the endogenous SRIF ligands but have differing distribution in various tissues. Somatostatin binds to the five distinct receptor (SSTR) subtypes with relatively high and equal affinity for each subtype.

There is evidence that SRIF regulates cell proliferation by arresting cell growth via SSTR1, 2, 4, and 5 subtypes (Buscail L, et al., 1995 Proc Natl Acad Sci USA 92: 1580-1584; Buscail L, et al., 1994 Proc Natl Acad Sci USA 91: 2315-2319; Florio T, et al., 1999 Mol Endocrinol 13: 24-37; Sharma K, et al., 1999 Mol Endocrinol 13: 82-90), or by inducing apoptosis via SSTR3 subtype (Sharma K, et al., 1996 Mol Endocrinol 10: 1688-1696). SRIF and various analogues have been shown to inhibit normal and neoplastic cell proliferation in vitro and in vivo (Lamberts S W, et al., Endocr Rev 12: 450-482) via specific SRIF receptors (SSTR's) (Patel Y C, 1999 Front Neuroendocrinology 20: 157-198) and possibly different postreceptor actions (Weckbecker G, et al., Pharmacol Ther 60: 245-264; Bell G I, Reisine T 1993 Trends Neurosci 16: 34-38; Patel Y C, et al., Biochem Biophys Res Commun 198: 605-612; Law S F, et al., Cell Signal 7:1-8). In addition, there is evidence that distinct SSTR subtypes are expressed in normal and neoplastic human tissues, conferring different tissue affinities for various SRIF analogues and variable clinical response to their therapeutic effects.

As is well known to those skilled in the art, SRIF and analogs are useful in the treatment of a great variety of diseases and/or conditions. An exemplary but by no means exhaustive list of such diseases and/or conditions would include: Cushings Syndrome (see Clark, R. V. et al, Clin. Res. 38, p. 943A, 1990); gonadotropinoma (see Ambrosi B., et al., Acta Endocr. (Copenh.) 122, 569-576, 1990); hyperparathyroidism (see Miller, D., et al., Canad. Med. Ass. J., Vol. 145, pp. 227-228, 1991); Paget's disease (see, Palmieri, G. M. A., et al., J. of Bone and Mineral Research, 7, (Suppl. 1), p. S240 (Abs. 591), 1992); VIPoma (see Koberstein, B., et al., Z. Gastroenterology, 28, 295-301, 1990 and Christensen, C., Acta Chir. Scand. 155, 541-543, 1989); nesidioblastosis and hyperinsulinism (see Laron, Z., Israel J. Med. Sci., 26, No. 1, 1-2, 1990, Wilson, D. C., Irish J. Med. Sci., 158, No. 1, 31-32, 1989 and Micic, D., et al., Digestion, 16, Suppl. 1.70. Abs. 193, 1990); gastrinoma (see Bauer, F. E., et al., Europ. J. Pharmacol., 183, 55 1990); Zollinger-Ellison Syndrome (see Mozell, E., et al., Surg. Gynec. Obstet., 170, 476-484, 1990); hypersecretory diarrhea related to AIDS and other conditions (due to AIDS, see Cello, J. P., et al., Gastroenterology, 98, No. 5, Part 2, Suppl., A163 1990; due to elevated gastrin-releasing peptide, see Alhindawi, R., et al., Can. J. Surg., 33, 139-142, 1990; secondary to intestinal graft vs. host disease, see Bianco J. A., et al., Transplantation, 49, 1194-1195, 1990; diarrhea associated with chemotherapy, see Petrelli, N., et al., Proc. Amer. Soc. Clin. Oncol., Vol. 10, P 138, Abstr. No. 417 1991); irritable bowel syndrome (see O'Donnell, L. J. D., et al., Aliment. Pharmacol. Therap., Vol. 4., 177-181, 1990); acute or chronic pancreatitis (see Tulassay, Z., et al., Gastroenterology, 98, No. 5, Part 2, Suppl., A238, 1990); Crohn's Disease (see Fedorak, R. N., et al., Can. J. Gastroenterology, 3, No. 2, 53-57, 1989); systemic sclerosis (see Soudah, H., et al., Gastroenterology, 98, No. 5, Part 2, Suppl., A129, 1990); thyroid cancer (see Modigliani, E., et al., Ann., Endocr. (Paris), 50, 483-488, 1989); psoriasis (see Camisa, C., et al., Cleveland Clinic J. Med., 57, No. 1, 71-76, 1990); hypotension (see Hoeldtke, R. D., et al., Arch. Phys. Med. Rehabil., 69, 895-898, 1988 and Kooner, J. S., et al., Brit. J. Clin. Pharmacol., 28, 735P-736P, 1989); panic attacks (see Abelson, J. L., et al., Clin. Psychopharmacol., 10, 128-132, 1990); sclerodoma (see Soudah, H., et al., Clin. Res., Vol. 39, p. 303A, 1991); small bowel obstruction (see Nott, D. M., et al., Brit. J. Surg., Vol. 77, p. A691, 1990); gastroesophageal reflux (see Branch, M. S., et al., Gastroenterology, Vol. 100, No. 5, Part 2 Suppl., p. A425, 1991); duodenogastric reflux (see Hasler, W., et al., Gastroenterology, Vol. 100, No. 5, Part 2, Suppl., p. A448, 1991); Graves' Disease (see Chang, T. C., et al., Brit. Med. J., 304, p. 158, 1992); polycystic ovary disease (see Prelevic, G. M., et al., Metabolism Clinical and Experimental, 41, Suppl. 2, pp 76-79, 1992); upper gastrointestinal bleeding (see Jenkins, S. A., et al., Gut., 33, pp. 404-407, 1992 and Arrigoni, A., et al., American Journal of Gastroenterology, 87, p. 1311, (abs. 275), 1992); pancreatic pseudocysts and ascites (see Hartley, J. E., et al., J. Roy. Soc. Med., 85, pp. 107-108, 1992); leukemia (see Santini, et al., 78, (Suppl. 1), p. 429A (Abs. 1708), 1991); meningioma (see Koper, J. W., et al., J. Clin. Endocr. Metab., 74, pp. 543-547, 1992); and cancer cachexia (see Bartlett, D. L., et al., Surg. Forum., 42, pp. 14-16, 1991).

Binding to the different types of somatostatin receptor subtypes have been associated with the treatment of various conditions and/or diseases. For example, the inhibition of growth hormone has been attributed to the somatostatin type-2 receptor (“SSTR-2”) see, e.g., Raynor, et al., Molecular Pharmacol. 43:838 (1993); Lloyd, et al., Am. J. Physiol. 268:G102 (1995); while inhibition of insulin attributed to the somatostatin type-5 receptor (“SSTR-5”), see, e.g., Coy, et al. 197:366-371 (1993). Other indications associated with activation of the SRIF receptor subtypes are inhibition of insulin and/or glucagon and more particularly diabetes mellitus, angiopathy, proliferative retinopathy, dawn phenomenon and nephropathy; inhibition of gastric acid secretion and more particularly peptic ulcers, enterocutaneous and pancreaticocutaneous fistula, Dumping syndrome, watery diarrhea syndrome, and gastrointestinal hormone secreting tumors; treatment of cancer such as hepatoma; inhibition of angiogenesis, treatment of inflammatory disorders such as arthritis; retinopathy; chronic allograft rejection; angioplasty; preventing graft vessel and gastrointestinal bleeding.

It is preferred to have an analog which is selective for the specific SRIF receptor subtype or subtypes responsible for the desired biological response, thus, reducing interaction with other receptor subtypes which could lead to undesirable side effects. Further, because of the short half-life of native SRIF, various SRIF analogs have been developed, e.g., for the treatment of acromegaly, (Raynor, et al., Molecular Pharmacol. 43:838 (1993)).

Activation of SRIF receptor subtypes 2 and 5 have been associated with growth hormone suppression and more particularly GH secreting adenomas (Acromegaly) and TSH secreting adenomas. Studies in cultured pituitary adenoma cells have demonstrated that SSTR subtype 2 and 5 act synergistically in the suppression of growth hormone and prolactin secretion (Shimon I, et al., 1997 J. Clinical Invest. 100:2386-2392, Jaquet P, et al., 2000 J Clin Endocrinol Metab. 85:781-792). Activation of type 2 but not type 5 has been associated with treating prolactin secreting adenomas. However, while pituitary adenomas are known to express SSTR's, the antiproliferative activity of SRIF analogues on tumor cells has not been clearly demonstrated (Mahler C, et al., Clin Endocrinol 33: 261-9; Lupoli G, et al., Cancer 78: 1114-8; Smid W M, et al., Neth J Med 40: 240-243).

Thus, development and assessment of SSTR subtype analogues selective on pituitary adenoma cell growth provides a useful tool for clinical application. The present invention relates to the discovery that the pituitary adenomas respond to SSTR-1, SSTR2 and SSTR5 activation by subtype selective agonists in terms of [³H]thy incorporation and cell number. Each of SSTR1, SSTR2 and SSTR5 subtype selective agonists significantly suppress [³H]thy incorporation, i.e., inhibit DNA synthesis, and reduce cell proliferation, with SSTR1 selective agonists demonstrating the most potent effect of the three. Further, SSTR2 preferential agonists administered in combination with SSTR5 preferential agonists results in a synergistic effect, resulting in a greater suppression of proliferation than what would be otherwise expected.

### SUMMARY OF THE INVENTION

In one aspect, the present invention is directed to a method of reducing the rate of proliferation of pituitary adenoma cells which method comprises contacting said pituitary adenoma cells with one or more of an SSTR1 agonist, and/or one or more of an SSTR2 agonist, and/or one or more of SSTR5 agonist, or one or more pharmaceutically acceptable salts thereof, either alone or in combination.

A preferred method of the foregoing aspect features contacting said pituitary adenoma cells with an SSTR1 preferential agonist. Another preferred method of the foregoing aspect features contacting said pituitary adenoma cells with an SSTR1 selective agonist, more preferably Caeg-c(D-Cys-Pal-D-Trp-Lys-D-Cys)-Thr(Bzl)-Tyr-NH₂, or a pharmaceutically acceptable salt thereof.

Another preferred method of the foregoing aspect a method features contacting said pituitary adenoma cells with an SSTR2 preferential agonist. Another preferred method of the foregoing aspect features contacting said pituitary adenoma cells with an SSTR2 selective agonist, more preferably D-Nal-cyclo[Cys-Tyr-D-Trp-Lys-Val-Cys]-Thr-NH₂, cyclo[Tic-Tyr-D-Trp-Lys-Abu-Phe], 4-(2-Hydroxyethyl)-1-piperazinylacetyl-D-Phe-cyclo(Cys-Tyr-D-Trp-Lys-Abu-Cys)-Thr-NH₂, or 4-(2-Hydroxyethyl)-1-piperazine-2-ethanesulfonyl-D-Phe-cyclo(Cys-Tyr-D-Trp-Lys-Abu-Cys)-Thr-NH₂, or a pharmaceutically acceptable salt thereof, wherein “4-(2-Hydroxyethyl)-1-piperazinylacetyl” denotes the structure:

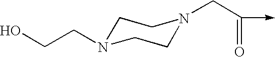

and “4-(2-Hydroxyethyl)-1-piperazine-2-ethanesulfonyl-” denotes the structure:

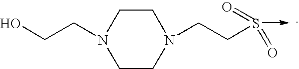

Yet another preferred method of the foregoing aspect a method features contacting said pituitary adenoma cells with an SSTR5 preferential agonist. Another preferred method of the foregoing aspect features contacting said pituitary adenoma cells with an SSTR5 selective agonist, more preferably D-Phe-Phe-Trp-D-Trp-Lys-Thr-Phe-Thr-NH₂, or a pharmaceutically acceptable salt thereof.

Still yet another preferred method of the foregoing aspect features contacting said pituitary adenoma cells with one or more of an SSTR2 preferential agonist together with one or more of an SSTR5 preferential agonist.

In one embodiment of the foregoing aspect said SSTR1 agonist comprises Caeg-c(D-Cys-Pal-D-Trp-Lys-D-Cys)-Thr(Bzl)-Tyr-NH₂, or a pharmaceutically acceptable salt thereof, said SSTR2 agonist comprises D-Nal-cyclo[Cys-Tyr-D-Trp-Lys-Val-Cys]-Thr-NH₂, cyclo[Tic-Tyr-D-Trp-Lys-Abu-Phe], 4-(2-Hydroxyethyl)-1-piperazinylacetyl-D-Phe-cyclo(Cys-Tyr-D-Trp-Lys-Abu-Cys)-Thr-NH₂, or 4-(2-Hydroxyethyl)-1-piperazine-2-ethanesulfonyl-D-Phe-cyclo(Cys-Tyr-D-Trp-Lys-Abu-Cys)-Thr-NH₂, or a pharmaceutically acceptable salt thereof, and said SSTR5 agonist comprises D-Phe-Phe-Trp-D-Trp-Lys-Thr-Phe-Thr-NH₂, or a pharmaceutically acceptable salt thereof.

In still yet another preferred method of the foregoing aspect said pituitary adenoma is a growth hormone-secreting adenoma, an ACTH-secreting adenoma, a prolactin-secreting adenoma, a TSH-secreting adenoma, a gonadotropin-secreting adenoma, a mixed secretion adenoma, or a non-functioning adenoma.

In another aspect the present invention is directed to a method of reducing secretion of one or more of growth hormone, ACTH, prolactin, TSH and/or gonadotropin in a patient in need of such reducing, said method comprising administering to said patient an effective amount of one or more of an SSTR1 agonist, and/or one or more of an SSTR2 agonist, and/or one or more of SSTR5 agonist, or one or more pharmaceutically acceptable salts thereof, either alone or in combination, wherein said effective amount is that amount which is effective to reduce said secretion.

In another aspect, the present invention is directed to a method of treating a patient suffering from adenoma which method comprises administering to said patient an effective amount of one or more of an SSTR1 agonist, and/or one or more of an SSTR2 agonist, and/or one or more of SSTR5 agonist, or one or more pharmaceutically acceptable salts thereof, either alone or in combination, wherein said effective amount is that amount which is effective to bring about the desired therapeutic effect.

A preferred method of this aspect of the invention features a method of treating pituitary adenoma which comprises administering to a patient in need thereof an effective amount of a SSTR1 agonist or a pharmaceutically acceptable salt thereof. In a preferred method of the immediately foregoing aspect said SSTR1 agonist comprises an SSTR1 selective agonist, or a pharmaceutically acceptable salt thereof.

Another preferred method of this aspect of the invention features a method of treating pituitary adenoma which comprises administering to a patient in need thereof an effective amount of a SSTR2 agonist or a pharmaceutically acceptable salt thereof. In a preferred method of the immediately foregoing aspect said SSTR2 agonist comprises an SSTR1 selective agonist, or a pharmaceutically acceptable salt thereof.

Another preferred method of this aspect of the invention features a method of treating pituitary adenoma which comprises administering to a patient in need thereof an effective amount of a SSTR5 agonist or a pharmaceutically acceptable salt thereof. In a preferred method of the immediately foregoing aspect said SSTR5 agonist comprises an SSTR5 selective agonist, or a pharmaceutically acceptable salt thereof.

In another preferred method of this aspect of the invention is features a method of treating pituitary adenoma which comprises administering to a patient in need thereof an effective amount of a SSTR2 agonist or a pharmaceutically acceptable salt thereof in combination with a SSTR5 agonist or a pharmaceutically acceptable salt thereof. In a preferred method of the immediately foregoing aspect said SSTR2 agonist comprises an SSTR2 selective agonist, or a pharmaceutically acceptable salt thereof and said SSTR5 agonist comprises an SSTR5 selective agonist or a pharmaceutically acceptable salt thereof.

In yet another preferred method, said SSTR1 agonist, and/or said SSTR2 agonist, and/or SSTR5 agonist each has, independently, a Ki value of less than 10 nM, more preferably less than 5 nM, even more preferably less than 1 nM, as determined by the receptor binding assay described herein.

In another aspect, the invention comprises a method of reducing the secretory activity of adenoma cells, which method comprises contacting said adenoma cells with one or more of an SSTR1 agonist, and/or one or more of an SSTR2 agonist, and/or one or more of SSTR5 agonist, or one or more pharmaceutically acceptable salts thereof, either alone or in combination. As will readily be appreciated, this method shares the fundamental features of the foregoing aspects of the invention.

### DETAILED DESCRIPTION OF THE INVENTION

It is believed that one skilled in the art can, based on the description herein, utilise the present invention to its fullest extent. The following specific embodiments are, therefore, to be construed as merely illustrative, and not limitative of the remainder of the disclosure in any was whatsoever.

Unless defined otherwise, all technical and scientific terms used herein have the same meaning as commonly understood by one of ordinary skill in the art to which this invention belongs. Also, all publications, patent applications, patents, and other references mentioned herein are incorporated by reference, each in its entirety.

A somatostatin agonist may be one or more of an SSTR-1 agonist, SSTR-2 agonist, SSTR-3 agonist, SSTR4 agonist or a SSTR-5 agonist.

What is meant by a somatostatin type-1 receptor agonist (i.e., SSTR-1 agonist) is a compound which (1) has a high binding affinity (e.g., Ki of less than 100 nM or preferably less than 10 nm or less than 1 nM) for SSTR-1 (e.g., as defined by the receptor binding assay described below) and (2) decreases the rate of proliferation of pituitary adenoma cells (e.g., as shown by the biological assay described below). What is meant by a somatostatin type-1 receptor selective agonist is a somatostatin type-1 receptor agonist which has a higher binding affinity (i.e., lower Ki) for SSTR-1 than for SSTR-2 or SSTR-5.

What is meant by a somatostatin type-2 receptor agonist (i.e., SSTR-2 agonist) is a compound which (1) has a high binding affinity (e.g., Ki of less than 100 nM or preferably less than 10 nm or less than 1 nM) for SSTR-2 (e.g., as defined by the receptor binding assay described below) and (2) decreases the rate of proliferation of pituitary adenoma cells (e.g., as shown by the biological assay described below). What is meant by a somatostatin type-2 receptor selective agonist is a somatostatin type-2 receptor agonist which has a higher binding affinity (i.e., lower Ki) for SSTR-2 than for SSTR-1 or SSTR-5.

What is meant by a somatostatin type-5 receptor agonist (i.e., SSTR-5 agonist) is a compound which (1) has a high binding affinity (e.g., Ki of less than 100 nM or preferably less than 10 nM or less than 1 nM) for SSTR-5 (e.g., as defined by the receptor binding assay described below) and (2) decreases the rate of proliferation of pituitary adenoma cells (e.g., as shown by the biological assay described below). What is meant by a somatostatin type-5 receptor selective agonist is a somatostatin type-5 receptor agonist which has a higher binding affinity (i.e., lower Ki) for SSTR-5 than for SSTR-1 or SSTR-2.

In one embodiment, the SSTR-1 agonist is also a SSTR-1 selective agonist. In another embodiment, the SSTR-1 selective agonist has a Ki value for SSTR-1 that is at least 2 times (e.g., at least 5 times or at least 10 times) lower than it has for the SSTR-2 receptor or the SSTR-5 receptor (e.g., as defined by the receptor binding assay described below).

In another embodiment, the SSTR-2 agonist is also a SSTR-2 selective agonist. In another embodiment, the SSTR-2 selective agonist has a Ki value for SSTR-2 that is at least 2 times (e.g., at least 5 times or at least 10 times) lower than it has for the SSTR-1 receptor or the SSTR-5 receptor (e.g., as defined by the receptor binding assay described below).

In still another embodiment, the SSTR-5 agonist is also a SSTR-5 selective agonist. In another embodiment, the SSTR-5 selective agonist has a Ki value for SSTR-5 that is at least 2 times (e.g., at least 5 times or at least 10 times) lower than it has for the SSTR-1 receptor or the SSTR-2 receptor (e.g., as defined by the receptor binding assay described below).

Examples of SSTR-1 agonists which may be used to practice the present invention include, but are not limited to Caeg-c(D-Cys-Pal-D-Trp-Lys-D-Cys)-Thr(Bzl)-Tyr-NH2, (Compound 1) having the following structure:

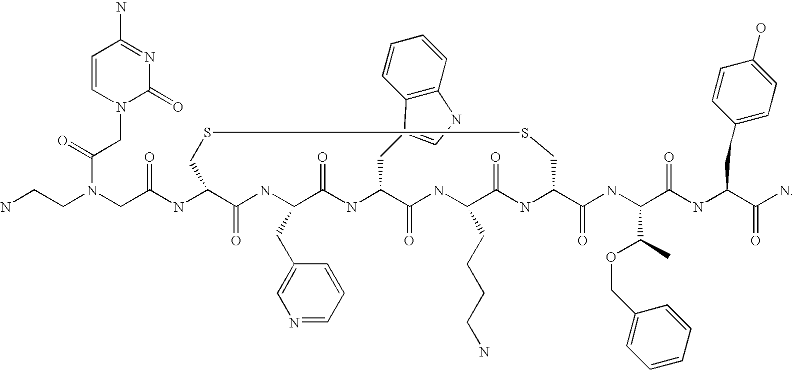

Examples of SSTR-2 agonists which may be used to practice the present invention include, but are not limited to:

D-Nal-cyclo[Cys-Tyr-D-Trp-Lys-Val-Cys]-Thr-NH₂, i.e., LANREOTIDE cyclo[Tic-Tyr-D-Trp-Lys-Abu-Phe], (Compound 2)

4-(2-Hydroxyethyl)-1-piperazinylacetyl-D-Phe-cyclo(Cys-Tyr-D-Trp-Lys-Abu-Cys)Thr-NH₂, and

4-(2-Hydroxyethyl)-1-piperazine-2-ethanesulfonyl-D-Phe-cyclo(Cys-Tyr-D-Trp-Lys-Abu-Cys)-Thr-NH₂.

An example of SSTR-5 agonist which may be used to practice the present invention includes, but is not limited to:

D-Phe-Phe-Trp-D-Trp-Lys-Thr-Phe-Thr-NH₂. (Compound 3)

Further examples of somatostatin agonists are those covered by formulae or those specifically recited in the publications set forth below, all of which are hereby incorporated by reference.

EP Application No. P5 164 EU (Inventor: G. Keri);

Van Binst, G. et al. Peptide Research 5:8 (1992);

Horvath, A. et al. Abstract, “Conformations of Somatostatin Analogs Having Antitumor Activity”, 22nd European peptide Symposium, Sep. 13-19, 1992, Interlaken, Switzerland;

PCT Application No. WO 91/09056 (1991);

EP Application No. 0 363 589 A2 (1990);

U.S. Pat. No. 4,904,642 (1990);

U.S. Pat. No. 4,871,717 (1989);

U.S. Pat. No. 4,853,371 (1989);

U.S. Pat. No. 4,725,577 (1988);

U.S. Pat. No. 4,684,620 (1987);

U.S. Pat. No. 4,650,787 (1987);

U.S. Pat. No. 4,603,120 (1986);

U.S. Pat. No. 4,585,755 (1986);

EP Application No. 0 203 031 A2 (1986);

U.S. Pat. No. 4,522,813 (1985);

U.S. Pat. No. 4,486,415 (1984);

U.S. Pat. No. 4,485,101 (1984);

U.S. Pat. No. 4,435,385 (1984);

U.S. Pat. No. 4,395,403 (1983);

U.S. Pat. No. 4,369,179 (1983);

U.S. Pat. No. 4,360,516 (1982);

U.S. Pat. No. 4,358,439 (1982);

U.S. Pat. No. 4,328,214 (1982);

U.S. Pat. No. 4,316,890 (1982);

U.S. Pat. No. 4,310,518 (1982);

U.S. Pat. No. 4,291,022 (1981);

U.S. Pat. No. 4,238,481 (1980);

U.S. Pat. No. 4,235,886 (1980);

U.S. Pat. No. 4,224,199 (1980);

U.S. Pat. No. 4,211,693 (1980);

U.S. Pat. No. 4,190,648 (1980);

U.S. Pat. No. 4,146,612 (1979);

U.S. Pat. No. 4,133,782 (1979);

U.S. Pat. No. 5,506,339 (1996);

U.S. Pat. No. 4,261,885 (1981);

U.S. Pat. No. 4,728,638 (1988);

U.S. Pat. No. 4,282,143 (1981);

U.S. Pat. No. 4,215,039 (1980);

U.S. Pat. No. 4,209,426 (1980);

U.S. Pat. No. 4,190,575 (1980);

EP Patent No. 0 389 180 (1990);

EP Application No. 0 505 680 (1982);

EP Application No. 0 083 305 (1982);

EP Application No. 0 030 920 (1980);

PCT Application No. WO 88/05052 (1988);

PCT Application No. WO 90/12811 (1990);

PCT Application No. WO 97/01579 (1997);

PCT Application No. WO 91/18016 (1991);

PCT Application No. WO 00/75186 (2000);

U.K. Application No. GB 2,095,261 (1981); and

French Application No. FR 2,522,655 (1983).

Note that for all somatostatin agonists described herein, each amino acid residue represents the structure of —NH—C(R)H—CO—, in which R is the side chain (e.g., CH₃ for Ala). Lines between amino acid residues represent peptide bonds which join the amino acids. Also, where the amino acid residue is optically active, it is the L-form configuration that is intended unless D-form is expressly designated. For clarity, disulfide bonds (e.g., disulfide bridge) which exist between two free thiols of Cys residues are not shown. Abbreviations of the common amino acids are in accordance with IUPAC-IUB recommendations.

### Synthesis of Somatostatin Agonists

The methods for synthesizing somatostatin agonists is well documented and are within the ability of a person of ordinary skill in the art.

Synthesis of short amino acid sequences is well established in the peptide art. For example, synthesis of H-D-Phe-Phe-Phe-D-Trp-Lys-Thr-Phe-Thr-NH₂, described above, can be achieved by following the protocol set forth in Example I of European Patent Application 0 395 417 A1. The synthesis of somatostatin agonists with a substituted N-terminus can be achieved, for example, by following the protocol set forth in WO 88/02756, European Patent Application No. 0 329 295, and PCT Publication No. WO 94/04752.

Some of the compounds of the instant invention can have at least one asymmetric center. Additional asymmetric centers may be present on the molecule depending upon the nature of the various substituents on the molecule. Each such asymmetric center will produce two optical isomers and it is intended that all such optical isomers, as separated, pure or partially purified optical isomers, racemic mixtures or diastereomeric mixtures thereof, are included within the scope of the instant invention.

The compounds of the instant invention generally can be isolated in the form of their pharmaceutically acceptable acid addition salts, such as the salts derived from using inorganic and organic acids. Examples of such acids are hydrochloric, nitric, sulfuric, phosphoric, formic, acetic, trifluoroacetic, propionic, maleic, succinic, D-tartaric, L-tartaric, malonic, methane sulfonic and the like. In addition, certain compounds containing an acidic function such as a carboxy can be isolated in the form of their inorganic salt in which the counter-ion can be selected from sodium, potassium, lithium, calcium, magnesium and the like, as well as from organic bases.

The pharmaceutically acceptable salts can be formed by taking about 1 equivalent of e.g., a SSTR-1 agonist, e.g., compound 1, and contacting it with about 1 equivalent or more of the appropriate corresponding acid of the salt which is desired. Work-up and isolation of the resulting salt is well-known to those of ordinary skill in the art.

The compounds of this invention can be administered by oral, parenteral (e.g., intramuscular, intraperitoneal, intravenous or subcutaneous injection, or implant), nasal, vaginal, rectal, sublingual or topical routes of administration and can be formulated with pharmaceutically acceptable carriers to provide dosage forms appropriate for each route of administration. Accordingly, the present invention includes within its scope pharmaceutical compositions comprising, as an active ingredient, at least one SSTR-2 agonist in association with a pharmaceutically acceptable carrier.

Solid dosage forms for oral administration include capsules, tablets, pills, powders and granules. In such solid dosage forms, the active compound is admixed with at least one inert pharmaceutically acceptable carrier such as sucrose, lactose, or starch. Such dosage forms can also comprise, as is normal practice, additional substances other than such inert diluents, e.g., lubricating agents such as magnesium stearate. In the case of capsules, tablets and pills, the dosage forms may also comprise buffering agents. Tablets and pills can additionally be prepared with enteric coatings.

Liquid dosage forms for oral administration include pharmaceutically acceptable emulsions, solutions, suspensions, syrups, the elixirs containing inert diluents commonly used in the art, such as water. Besides such inert diluents, compositions can also include adjuvants, such as wetting agents, emulsifying and suspending agents, and sweetening, flavoring and perfuming agents.

Preparations according to this invention for parenteral administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized by, for example, filtration through a bacteria-retaining filter, by incorporating sterilizing agents into the compositions, by irradiating the compositions, or by heating the compositions. They can also be manufactured in the form of sterile solid compositions which can be dissolved in sterile water, or some other sterile injectable medium immediately before use.

Compositions for rectal or vaginal administration are preferably suppositories which may contain, in addition to the active substance, excipients such as coca butter or a suppository wax.

Compositions for nasal or sublingual administration are also prepared with standard excipients well known in the art.

In general, an effective dosage of active ingredient in the compositions of this invention may be varied; however, it is necessary that the amount of the active ingredient be such that a suitable dosage form is obtained. The selected dosage depends upon the desired therapeutic effect, on the route of administration, and on the duration of the treatment, all of which are within the realm of knowledge of one of ordinary skill in the art. Generally, dosage levels of between 0.0001 to 100 mg/kg of body weight daily are administered to humans and other animals, e.g., mammals.

A preferred dosage range is 0.01 to 10.0 mg/kg of body weight daily, which can be administered as a single dose or divided into multiple doses.

SSTR Selective Agonists and Antagonists

Functional Assays

Each compound was resuspended in 0.01 N acetic acid containing 0.1% bovine serum albumin (BSA) in order to provide uniform solubility and prevent non-specific binding to the various preparation surfaces. Specificity and selectivity of the analogues were determined by Radioligand Binding Assay on CHO-K1 cells stably transfected with each of the SSTR subtypes, as follows.

The complete coding sequences of genomic fragments of the SSTR 1, 2, 3, and 4 genes and a cDNA clone for SSTR 5 were subcloned into the mammalian expression vector pCMV (Life Technologies, Milano, Italy). Clonal cell lines stably expressing SSTR's 1-5 were obtained by transfection into CHO-K1 cells (ATCC, Manassas, Va., USA) using the calcium phosphate co-precipitation method (Davis L, et al., 1994 In: Basic methods in Molecular Biology, 2nd edition, Appleton & Lange, Norwalk, Conn., USA: 611-646). The plasmid pRSV-neo (ATCC) was included as a selectable marker. Clonal cell lines were selected in RPMI 1640 media containing 0.5 mg/ml of G418 (Life Technologies, Milano, Italy), ring cloned, and expanded into culture.

Membranes for in vitro receptor binding assays were obtained by homogenizing the CHO-K1 cells expressing the SSTR's subtypes in ice-cold 50 mM Tris-HCl and centrifuging twice at 39000 g (10 min), with an intermediate resuspension in fresh buffer. The final pellets were resuspended in 10 mM Tris-HCl for assay. For the SSTR 1, 3, 4, and 5 assays, aliquots of the membrane preparations were incubated 90 min. at 25° C. with 0.05 nM [¹²⁵I-Tyr11]SRIF-14 in 50 mM HEPES (pH 7.4) containing 10 mg/ml BSA, 5 mM MgCl₂, 200 KIU/ml Trasylol, 0.02 mg/ml bacitracin, and 0.02 mg/ml phenylmethylsuphonyl fluoride. The final assay volume was 0.3 ml. For the SSTR 2 assay, 0.05 nM [¹²⁵I]MK-678 was employed as the radioligand and the incubation time was 90 min at 25° C. The incubations were terminated by rapid filtration through GF/C filters (pre-soaked in 0.3% polyethylenimine) using a Brandel filtration manifold. Each tube and filter were then washed three times with 5 ml aliquots of ice-cold buffer. Specific binding was defined as the total radioligand bound minus that bound in the presence of 1000 nM SRIF-14 for SSTR 1, 3, 4, and 5, or 1000 nM MK-678 for SSTR2.

Biological Activity Evaluation

Biological activity of SSTR selective agonists and antagonists was evaluated by the calcium mobilization assay in CHO-K1 cells expressing the human SSTR1, SSTR2 or SSTR5. The cells were harvested by incubating in a 0.3% EDTA/phosphate buffered saline solution (25° C.), and washed twice by centrifugation. The washed cells were resuspended in Hank's—buffered saline solution (HBSS) for loading of the fluorescent Ca²⁺ indicator Fura-2AM. Cell suspensions (approximately 10⁶ cells/ml) were incubated with 2 mM Fura-2AM for 30 min at 25° C. Unloaded Fura-2AM was removed by centrifugation twice in HBBS, and the final suspensions were transferred to a spectrofluorometer (Hitachi F-2000) equipped with a magnetic stirring mechanism and a temperature-regulated cuvette holder. After equilibration to 37° C., the SRIF analogues were added for measurement of intracellular Ca²⁺ mobilization. The excitation and emission wavelengths were 340 and 510 nm, respectively. The evaluation of intracellular Ca²⁺ mobilization demonstrated that the biological activity of each of the various analogues was in keeping with its receptor binding profile.

The ability of SRIF analogues with differing affinity and specificity for SSTR1, SSTR2 and SSTR 5 subtypes to influence cell proliferative activity may be assessed by considering [³H]thy incorporation, an indirect measure of DNA synthetic activity, and the number of viable cells.

DNA Synthesis

The effects of SSTR selective agonists and antagonists on pituitary adenoma cell DNA synthesis were assessed by determining the rate of [³H]thymidine incorporation, as previously described (Davis L, et al., 1994 In: Basic methods in Molecular Biology, 2nd edition, Appleton & Lange, Norwalk, Conn., USA: 611-646, degli Uberti E C, et al., 1991 J Clin Endocrinol Metab 72: 1364-1371).

Approximately 10⁵ pituitary cells from two functioning adenomas were plated in quadruplicate wells the day of surgery and treated the day after with SRIF and with Compounds 1, 2, 3, and LANREOTIDE at 10⁻⁹M. After 48 h incubation in a medium supplemented with 10% FBS in the presence of [³H]thy (1.5 μCi/mL; 87 Ci/mmol) with or without each SRIF analogue. Treatments were renewed by adding fresh analogues to the wells after the first 24 h of incubation, without removing the medium.

After incubation, the cells were washed three times with ice-cold PBS and twice with 10% ice-cold trichloroacetic acid (TCA). TCA-precipitated material was solubilized in 500 μL 0.2 mol/L sodium hydroxide and 0.1% SDS. Cell-associated radioactivity was then counted in a scintillation spectrometer. Results (counts per min per well) were obtained by determining the mean value of at least six experiments in quadruplicate. The viability of cells in control and treated cultures was evaluated by Trypan blue staining.

Cell Proliferation

The effects of SSTR selective agonists on pituitary adenoma cell proliferation were assessed by the CELLTITER 96 Aqueous Non-Radioactive Cell Proliferation Assay (Promega, Milano, Italy), a colorimetric method for determining the number of viable cells in proliferation assays. The assay contains solutions of a tetrazolium compound (Owen's reagent; MTS) and an electron coupling reagent (phenazine methosulphate; PMS). MTS is bioreduced by cells into a formazan that is soluble in tissue culture medium. The absorbance of the formazan at 490 nm can be measured directly from 96 well assay plates (Zatelli M C, et al., 2000 J Clin Endocrinol Metab 85: 847-852; Cory A H, et al., 1991 Cancer Commun 3: 207-212). The conversion of MTS into the aqueous soluble formazan is accomplished by dehydrogenase enzymes found in metabolically active cells. The quantity of formazan product as measured by the amount of 490 nm absorbance is directly proportional to the number of living cells in culture.

Briefly, adenoma cells were plated in 96-multiwell plates (2×10⁴ cells/well) and incubated for 48 hours in a medium supplemented with 10% FBS in the presence or absence of each SRIF analogue (including in one instance, compounds 2 & 3 together) at a concentration of 10⁻⁹ M. Treatments were renewed by adding fresh analogues to the wells after the first 24 hours of incubation. At the end of the incubation period, 20 μl of a combined MTS/PMS solution was added to each well with a repeating pipette, and the plates were incubated for an additional 4 hours at 37° C. in a humidified 5% CO₂ atmosphere. The absorbance at 490 nm was then recorded using an ELISA plate reader (EASIA Reader, Medgenix). Results (absorbance at 490 nm) were obtained by determining the mean value of at least six experiments in eight replicates.

TABLE 1
[³H]Thy incorporation values for SRIF, LANREOTIDE, SSTR1
(compound 1), SSTR2 (compound 2) and SSTR5 (compound 3)
preferential agonists.

| | % Reduction | % Reduction |
|---|---|---|
| Compound | [³H]Thymidine | Viable Cells |
| SRIF | 10. | 8. |
| LANREOTIDE | 25. | 12. |
| Compound 1 | 31.5 | 28.5 |
| Compound 2 | 25. | 12.5 |
| Compound 3 | 15.5 | 18.5 |
| Compounds 2 + 3 | 68 | 32.5 |

It is to be understood that while the invention has been described in conjunction with the examples and the detailed description thereof, that the foregoing description is intended to illustrate and not limit the scope of the invention defined by the appended claims. Other aspects, advantages, and modifications are within the claims.

1. A method of reducing the rate of proliferation of pituitary adenoma cells which method comprises contacting said pituitary adenoma cells with one or more of an SSTR1 agonist, and/or one or more of an SSTR2 agonist, and/or one or more of an SSTR5 agonist, or one or more pharmaceutically acceptable salts thereof, either alone or in combination.

2. The method according to claim 1, comprising contacting said pituitary adenoma cells with an SSTR1 preferential agonist.

3. The method according to claim 1, comprising contacting said pituitary adenoma cells with an SSTR1 selective agonist.

4. The method according to claim 1, wherein said SSTR1 agonist has a Ki of less than 10 nM or less than 1 nM for SSTR-1.

5. The method according to claim 1, comprising contacting said pituitary adenoma cells with an SSTR2 preferential agonist.

6. The method according to claim 1, comprising contacting said pituitary adenoma cells with an SSTR2 selective agonist.

7. The method according to claim 1, wherein said SSTR2 agonist has a Ki of less than 10 nM or less than 1 nM for SSTR-2.

8. The method according to claim 1, comprising contacting said pituitary adenoma cells with an SSTR5 preferential agonist.

9. The method according to claim 1, comprising contacting said pituitary adenoma cells with an SSTR5 selective agonist.

10. The method according to claim 1, wherein said SSTR5 agonist has a Ki of less than 10 nM or less than 1 nM for SSTR-5.

11. The method according to claim 1, comprising contacting said pituitary adenoma cells with one or more of an SSTR2 preferential agonist and one or more of an SSTR5 preferential agonist.

12. The method according to claim 1, wherein said SSTR1 agonist is Caeg-c(D-Cys-Pal-D-Trp-Lys-D-Cys)-Thr(Bzl)-Tyr-NH₂, said SSTR2 agonist is D-Nal-cyclo[Cys-Tyr-D-Trp-Lys-Val-Cys]-Thr-NH₂, cyclo[Tic-Tyr-D-Trp-Lys-Abu-Phe], 4-(2-Hydroxyethyl)-1-piperazinylacetyl-D-Phe-cyclo(Cys-Tyr-D-Trp-Lys-Abu-Cys)-Thr-NH₂, or 4-(2-Hydroxyethyl)-1-piperazine-2-ethanesulfonyl-D-Phe-cyclo(Cys-Tyr-D-Trp-Lys-Abu-Cys)-Thr-NH₂, and said SSTR5 agonist is D-Phe-Phe-Trp-D-Trp-Lys-Thr-Phe-Thr-NH₂, or a pharmaceutically acceptable salt thereof.

13. The method according to claim 1, wherein said pituitary adenoma is a growth hormone-secreting adenoma, an ACTH-secreting adenoma, a prolactin-secreting adenoma, a TSH-secreting adenoma, a gonadotropin-secreting adenoma, a mixed secretion adenoma, or a non-functioning adenoma.

14. A method of treating pituitary adenoma which comprises administering to a patient in need thereof an effective amount of an SSTR1 agonist or a pharmaceutically acceptable salt thereof.

15. The method according to claim 14, wherein said SSTR1 agonist comprises an SSTR1 selective agonist, or a pharmaceutically acceptable salt thereof.

16. A method of treating pituitary adenoma which comprises administering to a patient in need thereof an effective amount of an SSTR2 agonist or a pharmaceutically acceptable salt thereof.

17. The method according to claim 16, wherein said SSTR2 agonist comprises an SSTR2 selective agonist, or a pharmaceutically acceptable salt thereof.

18. A method of treating pituitary adenoma which comprises administering to a patient in need thereof an effective amount of an SSTR5 agonist or a pharmaceutically acceptable salt thereof.

19. The method according to claim 18, wherein said SSTR5 agonist comprises an SSTR5 selective agonist, or a pharmaceutically acceptable salt thereof.

20. A method of treating pituitary adenoma which comprises administering to a patient in need thereof an effective amount of an SSTR2 agonist or a pharmaceutically acceptable salt thereof in combination with an SSTR5 agonist or a pharmaceutically acceptable salt thereof.

21. The method according to claim 20, wherein said SSTR2 agonist comprises an SSTR2 selective agonist and said SSTR5 agonist comprises an SSTR5 selective agonist.

22. The method according to claim 14, wherein said SSTR1 agonist is Caeg-c(D-Cys-Pal-D-Trp-Lys-D-Cys)-Thr(Bzl)-Tyr-NH₂, or a pharmaceutically acceptable salt thereof.

23. The method according to claim 17, wherein said SSTR-2 selective agonist is a compound selected from the group consisting of:
D-Nal-cyclo[Cys-Tyr-D-Trp-Lys-Val-Cys]-Thr-NH₂;
cyclo[Tic-Tyr-D-Trp-Lys-Abu-Phe];
4-(2-Hydroxyethyl)-1-piperazinylacetyl-D-Phe-cyclo(Cys-Tyr-D-Trp-Lys-Abu-Cys)-Thr-NH₂;
4-(2-Hydroxyethyl)-1-piperazine-2-ethanesulfonyl-D-Phe-cyclo(Cys-Tyr-D-Trp-Lys-Abu-Cys)-Thr-NH₂; and
pharmaceutically acceptable salts thereof.

24. The method according to claim 19, wherein said SSTR5 selective agonist is D-Phe-Phe-Trp-D-Trp-Lys-Thr-Phe-Thr-NH₂, or a pharmaceutically acceptable salt thereof.

25. A method of reducing secretion of one or more of growth hormone, ACTH, prolactin, TSH and/or gonadotropin in a patient in need of such reducing, said method comprising administering to said patient an effective amount of one or more of an SSTR1 agonist, and/or one or more of an SSTR2 agonist, and/or one or more of an SSTR5 agonist, or one or more pharmaceutically acceptable salts thereof, either alone or in combination, wherein said effective amount is an amount which is effective to reduce said secretion.

26. A method of treating a patient suffering from adenoma which method comprises administering to said patient an effective amount of one or more of an SSTR1 agonist, and/or one or more of an SSTR2 agonist, and/or one or more of an SSTR5 agonist, or one or more pharmaceutically acceptable salts thereof, either alone or in combination, wherein said effective amount is an amount which is effective to bring about the desired therapeutic effect.
